(19) United States
(12) Patent Application Publication
(10) Pub. No.: US 2009/0181071 A1
(43) Pub. Date: Jul. 16, 2009

(54) Oral Care Delivery System with Microcrystalline Wax

(76) Inventors: Samuel James St. John, Cincinnati, OH (US); Jayanth Rajaiah, Loveland, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/351,225

(22) Filed: Jan. 9, 2009

Related U.S. Application Data
(60) Provisional application No. 61/010,836, filed on Jan. 11, 2008.

(51) Int. Cl. A61K 8/02; A61K 8/22

(52) U.S. Cl. 424/443; 424/49; 424/53

(57) ABSTRACT

An oral care delivery system comprises a flexible substrate with a first side and a second side, with at least one oral care composition that is disposed adjacent the first side of the substrate, and at least one delivery layer that is disposed adjacent the second side of the substrate. A first delivery layer comprises a microcrystalline wax having a melting point between about 70 and about 95 degrees Celsius. The delivery system is applied to the surfaces of a plurality of teeth.

### CROSS REFERENCE TO RELATED APPLICATIONS

This application claims the benefit of U.S. Provisional Application No. 61/010,836, filed Jan. 11, 2008.

### FIELD OF THE INVENTION

The present invention relates to oral care delivery systems using a microcrystalline wax, and more particularly, to tooth whitening substances that include a microcrystalline wax.

### BACKGROUND OF THE INVENTION

Tooth whitening has become very popular over the past few years, with more and more consumers choosing to whiten their teeth. Options for tooth whitening include toothpastes, mouth rinses, chewing gums, in-office bleaching, and tooth whitening solutions used with a tray obtained either over-the-counter or from a dentist.

Tooth whitening products using a strip of material in combination with a chemical whitening agent are known in the art. For example, U.S. Pat. Nos. 5,891,453 and 5,879,691, the substances of which are incorporated herein by reference, describe a whitening product comprising a flexible strip of material and a tooth whitening composition with a peroxide agent and carboxypolymethylene gelling agent. While peroxide and carboxypolymethylene are common ingredients in tooth whitening applications, one or both can contribute to an undesirable taste sensation during use. As such, there is a continuing need for tooth whitening products that can deliver aesthetic agents that improve the oral experience.

Furthermore, U.S. Pat. No. 6,916,463, the substance of which is incorporated herein by reference, describes using a carrier material such as a wax to deliver an aesthetic agent. But the use of many types of wax can result in flaking, either in the product packaging or in the user's mouth, accelerated peroxide instability because of product incompatibility, and/or aesthetic agent adhesion to the packaging. Therefore, there is a further desire for products that can deliver flavor, aesthetic agents, and other oral care actives while minimizing any unwanted effects.

### SUMMARY OF THE INVENTION

An oral care delivery system is provided. The oral care delivery system comprises a flexible substrate with a first side and a second side, with at least one oral care composition that is disposed adjacent the first side of the substrate, and at least one delivery layer that is disposed adjacent the second side of the substrate. The first delivery layer comprises a microcrystalline wax having a melting point between about 70 and about 95 degrees Celsius. The delivery system is applied to the surfaces of a plurality of teeth.

### BRIEF DESCRIPTION OF THE DRAWINGS

While the specification concludes with claims which particularly point out and distinctly claim the present invention, it is believed that the present invention will be better understood from the following description of preferred embodiments, taken in conjunction with the accompanying drawings, in which like reference numerals identify identical elements and wherein:

FIG. 1 is a perspective view of a substantially flat substrate coated with an oral care composition for treating teeth and/or gums;

FIG. 2 is a cross-sectional view of the substrate of FIG. 1, taken along section line 2-2 of FIG. 1;

FIG. 3 is a cross-sectional view showing an alternative embodiment of the present invention, wherein a plurality of pockets are shown;

FIG. 4 is a perspective view of the substrate of FIG. 1 further including a release liner;

FIG. 5 is a cross-sectional view of the substrate of FIG. 1 taken along line 5-5 thereof,

FIG. 6 is a cross-sectional plan view of a plurality of teeth having the substrate of FIG. 1 applied thereto, wherein the substrate is applied to the front or labial surface of the teeth;

FIG. 7 is a cross-sectional side view of the teeth and the substrate of FIG. 6, taken along line 7-7 thereof,

FIG. 8 is a cross-sectional plan view of a plurality of teeth having the substrate of FIG. 1 applied thereto, wherein the substrate is applied to the front or labial surfaces of the teeth and the rear or lingual surfaces of the teeth and soft tissue adjacent the labial surfaces of the teeth;

FIG. 9 is a cross-sectional plan view of a plurality of teeth and substrate of FIG. 8, taken along line 9-9 thereof;

FIG. 10 is a cross-sectional view showing an alternative embodiment of the present invention, wherein there are two delivery layers; and

FIG. 11 is a schematic illustration of a process for manufacturing the embodiments of the present invention.

### DETAILED DESCRIPTION OF THE INVENTION

The abbreviation “cm”, as used herein, means centimeter. The abbreviation “mm” as used herein, means millimeter. The abbreviation “dmm” as used herein, means decimillimeter.

As used herein, the term “substrate” means an underlying layer, including but not limited to a strip, film, barrier, mesh, web, scrim, net, or weave. The substrate may be permanently or non-permanently deformable. The substrate may be pre-formed prior to use, such as in the shape of a dental arch or a tray.

As used herein, the term “nonbioerodible” means that a material maintains its physical integrity during and after use, such that it can be easily removed by a wearer after use by peeling it off.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

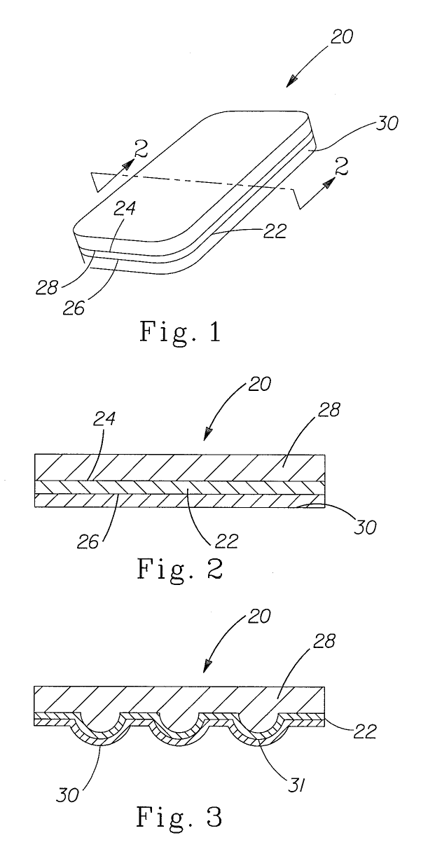

Referring to FIGS. 1, 2, and 3, an exemplary oral care delivery system 20 made in accordance with the present invention will now be described. The oral care delivery system 20 comprises a flexible substrate 22 having a first side 24 and a second side 26. Disposed adjacent the first side 24 is an oral care composition layer 28. The oral care composition layer 28 may comprise one or more oral care compositions. Disposed adjacent the second side 26 is a delivery layer 30.

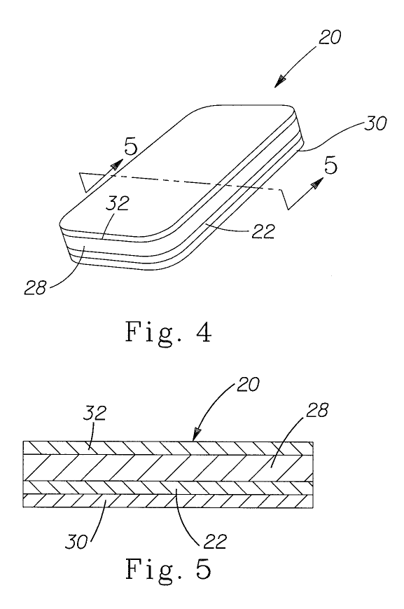

Optionally, an array or plurality of substantially unshaped pockets 31 can be formed in the substrate 22. The pockets are filled with the at least one oral care composition and provide a texture to the substrate. Since the pockets are formed in the substrate, the array of pockets is generally planar in nature. Also optionally, one or both sides of the oral care composition layer and the delivery layer that are opposite the substrate 22 can be covered by a release liner 32, as shown by way of example in FIGS. 4 and 5, or one or both sides of the oral care composition layer and the delivery layer can be exposed.

In some embodiments, the oral care composition layer 28 comprises a tooth whitening agent. In some embodiments, the tooth whitening agent is a peroxide agent.

In some embodiments, the delivery layer 30 comprises a microcrystalline wax. In some embodiments, the delivery layer may further comprise paraffin wax, an aesthetic agent, and/or at least one oral care active. In some embodiments, the microcrystalline wax is in contact with the surface of the substrate. In other embodiments, the delivery layer may be nonaqueous.

In still other embodiments, there may be more than one delivery layer. For example, as shown in FIG. 10, a second delivery layer 44 may be disposed adjacent the first delivery layer 30. The second delivery layer 44 may comprise a sweetening agent.

### Tooth Whitening Composition

While the present invention will be described with respect to a tooth whitening composition, it will be appreciated that the at least one oral care composition disposed adjacent the first side of the substrate can be provided in other forms with other oral care agents. For example, phosphates, fluoride ion sources, anti-microbial agents, anti-inflammatory agents, nutrients, enzymes, anti-oxidants, H-2 antagonists, and mixtures thereof may be used in combination with or in place of a tooth whitening agent. These and other oral care actives that are suitable for use with the present invention are described in U.S. Pat. No. 6,136,297, the substance of which is incorporated herein by reference.

The tooth whitening composition can be coated or spread on the substrate 22, and it forms a layer 28 having a thickness at least about 0.01 mm, or at least about 0.02 mm, or at least about 0.05 mm, or at least about 0.07 mm and/or less than about 0.05 mm, or less than about 1 mm, or less than about 2 mm, or less than about 3 mm. These measurements are taken by measuring from the surface of the substrate 22 and up through the oral care composition layer 28. While it is desirable for the oral care composition layer 28 to be a homogeneous, uniform and continuous layer, the layer 28 may also be non-uniform, non-continuous, and/or heterogeneous. For example, the oral care composition layer 28 can be a laminate or separated layers of components, an amorphous mixture of components, separate stripes or spots or other patterns of different components, or a combination of these structures. Further, the oral care composition layer can be formed as part of or intermixed with the substrate 22.

Alternatively, the at least one oral care composition can be substituted for the substrate 22, such as described by way of example in U.S. Pat. No. 6,419,906. For example, the oral care composition could be provided in the form of a film or strip comprising a water hydratable ethylene oxide polymer having a tooth whitening agent incorporated therein. The delivery layer 30 incorporating the microcrystalline wax and an aesthetic agent could then be formed as a film adjacent one side of the ethylene oxide polymer film.

As used herein, the phrase “disposed adjacent” is intended to refer to placement directly on the subject surface or it can include placement near the subject surface such as where there is an intermediate additional coating. For example, there could be a coating material disposed between the delivery layer 30 incorporating the microcrystalline wax and the aesthetic agent and the ethylene oxide polymer film.

The tooth whitening composition that forms the oral care composition layer 28 can be provided in the form of a viscous liquid, paste, gel, solution, solid, or any other state or phase that can form a layer. In one embodiment, the tooth whitening composition is provided in the form of a gel and has a viscosity between about 200 and about 1,000,000 cps at low shear rates (approximately one seconds⁻¹), and in another embodiment the viscosity is between about 100,000 and about 800,000 cps. In other embodiments, the viscosity is between about 150,000 and about 700,000 cps or between about 300,000 and about 700,000 cps.

The amount of tooth whitening composition will vary depending upon the intended use, the size of the substrate 22, concentration of the peroxide agent, and the desired benefit. Generally, less than about 1 gram is provided. In another embodiment, from about 0.05 grams to about 0.5 grams are provided and in yet another embodiment from about 0.1 gram to about 0.4 grams of the tooth whitening composition are provided. The amount of tooth whitening composition per square cm of substrate 22 is less than about 0.2 grams/cm², or from about 0.005 to about 0.1 grams/cm², or from about 0.01 grams/cm² to about 0.05 grams/cm².

The tooth whitening composition may also have a yield stress. Yield stress is the amount of force on a material before the material begins to move. The yield stress may be high enough so that the tooth whitening composition is able to form a thin layer and also to handle the disturbances caused by manufacturing, handling, and storage. The yield stress of the tooth whitening composition may be between about 2 Pascals and about 3000 Pascals, or between about 20 Pascals and about 2000 Pascals, or between about 200 Pascals and about 1500 Pascals, or between about 400 Pascals and about 1200 Pascals.

The tooth whitening or bleaching agents suitable for use with the tooth whitening composition include peroxides, metal chlorites, perborates, peroxyacids, and combinations thereof. Peroxide agents can include hydrogen peroxide, calcium peroxide, carbamide peroxide, and mixtures thereof. Suitable metal chlorites include calcium chlorite, barium chlorite, magnesium chlorite, lithium chlorite, sodium chlorite, and potassium chlorite. Additional tooth whitening agents include hypochlorite and chlorine dioxide. While the tooth whitening agent can be present in any concentration, it is preferred that the peroxide agent is present in an concentration of hydrogen peroxide equivalent of at least about 0.01%, or at least about 0.1%, or at least about 0.5%, or at least about 5%, or at least about 8%, or at least about 10%, or at least about 12%, or at least about 15% and/or less than about 15%, or less than about 20%, or less than about 25%, or less than about 30% or less than about 40% by weight of the tooth whitening composition. It is understood that these concentrations are expressed for hydrogen peroxide and appropriate conversions must be made for other peroxide liberating molecules such as carbamide peroxide, calcium peroxide, etc.

Additional constituents of the tooth whitening composition can include, but are not limited to, water, gelling agents, humectants, pH adjusting agents, stabilizing agents, desensitizing agents, and accelerating agents or bleach activators. A common gelling agent is a swellable polymer. An effective concentration of a gelling agent to enable the tooth whitening composition to form a layer will vary with each type of gelling agent. The thin layer may have a viscosity and yield stress enabling the tooth whitening composition to form the thin layer on the substrate. The tooth whitening composition formed with these agents may also provide sufficient adhesive attachment of substrate to the targeted area of the mouth. For example, the level of gelling agent to form the tooth whitening composition with a carboxypolymethylene, or other gelling agent such as polyvinyl pyrrolidone (PVP), is between about 0.1% and about 80%, or between about 1% and about 40%, or between about 2% and about 20%, or between about 3% and about 10%, by weight of the tooth whitening composition. An effective concentration of a poloxamer gelling agent is between about 10% and about 40%, or between about 20% and about 35%, or between about 25% and about 30%, by weight of the tooth whitening composition.

Suitable gelling agents useful in the present invention include “Pemulen” made by Noveon, Inc., carboxypolymethylene, PVP, carboxymethyl cellulose, carboxypropyl cellulose, hydroxyethyl cellulose, poloxamer, Laponite, carrageenan, Veegum, carboxyvinyl polymers, and natural gums such as gum karaya, xanthan gum, Guar gum, gum arabic, gum tragacanth, and mixtures thereof. Other usable gelling agents include gums such as algin, alginic acid, alginate salts, camitine, dextrin (starch gum), gellan gum, irish moss, tara gum, okra gum, acacia gum, amylopectin, pectina or pectin, ghatti gum, natto gum, sclerotium gum, kelp, locust bean gum, psyllium seed, tamarind gum, destria gum, chitosan, esters thereof (such as hydroxypropyl chitosan and hydroxypropyl guar), salts thereof (such as ammonium alginate, amylopectin, calcium alginate, calcium carrageenan, guar hydroxypropyltrimonium), and mixtures thereof. The preferable gelling agent for use in the present invention is carboxypolymethylene, obtained from B. F. Goodrich Company under the tradename “Carbopol”. Particularly preferable Carbopols include Carbopol 934, 940, 941, 956, 971, 974, 980, and mixtures thereof. Particularly preferred is Carbopol 956. Carboxypolymethylene is a slightly acidic vinyl polymer with active carboxyl groups.

Other polymers suitable for use with the present invention include ethylene oxide polymers, homopolymers or mixtures of ethylene oxide polymers of varying molecular weight ranging from about 10,000 Daltons and up to about 10,000,000 Daltons and preferably in the range of about 100,000 to about 1,500,000 Daltons. Such ethylene oxide polymers are commercially available from various sources. Polyethylene oxide in the molecular weight range of 10,000 to 1,000,000 Daltons is available from the Dow Chemical Company under the tradename POLYOX™. Other suitable polymers include polypropylene oxide, cross-linked polyacrylic acid, e.g., Carbopol, linear polyacrylic acid, polyvinyl alcohol, sodium alginate, methyl methacrylate, pullulan, agar, celluloses (e.g., hydroxypropylcellulose, hydroxyethyl cellulose, hydroxypropylmethyl cellulose, propylcellulose, ethyl cellulose, and hydroxymethyl cellulose), polyethylene oxide polypropylene oxide copolymers (e.g., poloxamer), microcrystalline cellulose, polyvinyl pyrolidone polyvinyl acetate copolymers, poly vinyl ester-methyl_copolymers, polyoxyethelene-polyoxypropylene block copolymer, and mixtures thereof.

Another possible adhesive suitable for use in the instant composition is polyvinylpyrrolidone with a molecular weight of about 50,000 to about 300,000. Still another possible adhesive suitable for use in the instant composition is a combination of Gantrez and the semisynthetic, water-soluble polymer carboxymethyl cellulose.

A pH adjusting agent may also be added to make the composition safe for oral tissues. These pH adjusting agents, or buffers, can be any material which is suitable to adjust the pH of the composition. Suitable materials include sodium bicarbonate, sodium phosphate, sodium hydroxide, ammonium hydroxide, potassium hydroxide, sodium stannate, triethanolamine, citric acid, hydrochloric acid, sodium citrate, and combinations thereof. The pH adjusting agents are added in sufficient concentrations so as to adjust the pH of the composition to between about 3 and about 10, or between about 4 and about 8.5, or between about 4.5 and about 8. The pH adjusting agents are generally present in a concentration between about 0.01% and about 15% or between about 0.05% and about 5%, by weight of the composition.

Suitable stabilizing agents include benzoic acid, salicylic acid, butylated hydroxytoluene, tin salts, phosphates, and others. Suitable bleach activators include trichloroisocyanuric acid and the phosphates, such as tetrasodium pyrophosphate.

Desensitizing agents may also be used in the tooth whitening composition. These agents may be preferred for consumers who have sensitive teeth. Desensitizing agents include potassium nitrate, citric acid, citric acid salts, strontium chloride, and combinations thereof. Potassium nitrate is a preferred desensitizing agent. Other agents which provide the benefit of reduced tooth sensitivity are also included in the present invention. Typically, the concentration of a desensitizing agent is between about 0.01% and about 10%, or between about 0.1% and about 8%, or between about 1% and about 7% by weight of the tooth whitening composition.

For a tooth whitening composition, it is often desirable to include a humectant or plasticizer as a constituent. A humectant provides Theological and/or physical stability and provides various aesthetics for a user. Common humectants include polyols (e.g., glycerin, sorbitol, polyethylene glycol, propylene glycol). The polyol may be present in a concentration of less than about 40%, or between about 0% and about 35%, or between about 1% and about 30%, or between about 5% and about 15%, by weight of the tooth whitening composition. As the concentration of polyol decreases, balance of the tooth whitening composition can comprise water. Generally, the concentration of water may be at least about 0%, or at least about 25%, or at least about 50%, or at least about 60%, or at least about 70% and/or less than about 99%, or less than about 90%, or less than about 80%, or less than about 70% by weight of the total tooth whitening composition. This concentration of water includes the free water that is added plus that amount that is introduced with other materials.

### Substrate

The substrate 22 serves as a protective barrier for the oral care composition. It prevents some or substantial leaching and/or erosion of the at least one oral care composition by, for example, the wearer's lips, tongue, as well as saliva. This allows the active in the at least one oral care composition to act upon the oral surface for an extended period of time, from several minutes to several hours. The term “act upon” is herein defined as bringing about a desired change. For example, if an oral care active is an anti-microbial substance, it reduces or eliminates proliferation of microbial growth that has an overall positive impact on the oral cavity including teeth and gingival tissue.

The substrate may comprise polymers, natural and synthetic woven materials, non-woven material, foil, paper, rubber, and combinations thereof. The substrate may be a single layer of material or a laminate of more than one layer. In one embodiment, the substrate is provided in the form of a substantially flat or planar strip of material. Suitable polymers include, but are not limited to, polyolefins such as polyethylene or polypropylene, ethylvinylacetate, polyesters, ethylvinyl alcohol and combinations thereof. Examples of polyesters include Mylar® and fluoroplastics such as Teflon®, both manufactured by DuPont. Alternatively, it is contemplated that the substrate 22 can be provided in other forms, such as preformed dental trays or flexible dental trays, wax, foams, hydratable films, porous webs or films, and combinations of any of the foregoing. Some of these other substrates are described in U.S. Pat. Nos. 6,419,906; 4,173,243; 5,310,563; 6,045,811; 5,326,685; 5,575,654; and RE 34,196, the substances of which are incorporated herein by reference.

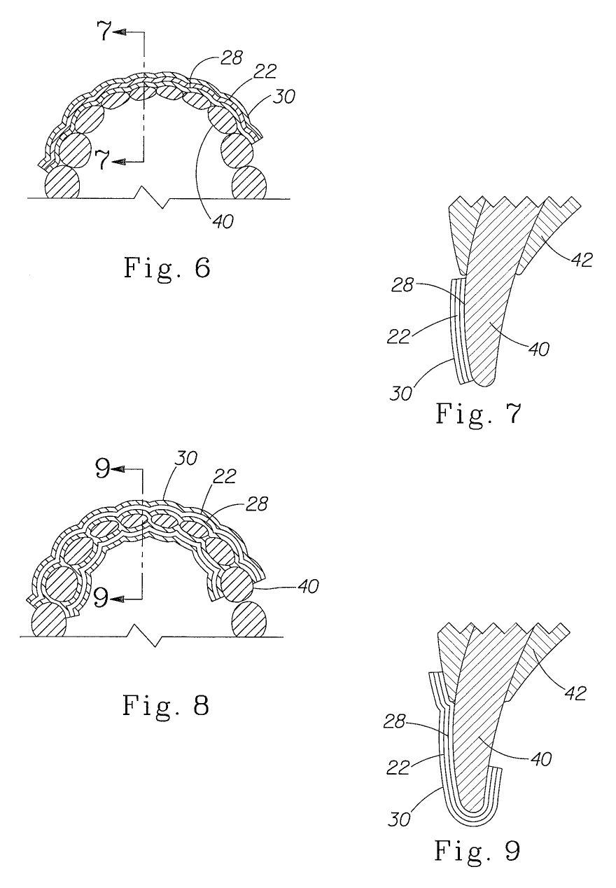

While the substrate 22 can be sized according to its application and is generally sized for the oral cavity of a human user and more particularly, in the tooth whitening product 20, the substrate is sized to individually fit the tooth or row of teeth 40 desired to be bleached, as shown generally in FIGS. 6, 7, 8, and 9. Generally, this is the front, six to eight teeth of the upper or lower rows of teeth that are visible when the wearer is smiling or either the maxillary dentition or the mandibular dentition. Optionally, the substrate 22 may fit the entire upper or lower rows of teeth when positioned against the teeth. In one embodiment, the substrate 22 is sized to cover a portion of labial surface (i.e., front surface) and the soft tissue 42 adjacent the teeth and fold over the incisal edge of the teeth and onto at least a portion of the lingual surface (i.e., back surface) of the teeth. In another embodiment, the substrate 22 is further sized to cover at least the central six anterior teeth (canine/cuspid to canine/cuspid). The substrate 22 can be a maxillary strip which is rectangular with rounded corners and measures approximately 6.5 cm long×1.5 cm wide and/or the substrate 22 can be a mandibular strip which is trapezoidal with rounded corners and measures 5 cm long×2 cm wide. Further description of the size and shape of the substrate 22 in a tooth whitening application is disclosed in U.S. patent application Ser. No. 09/268,185 filed Mar. 15, 1999, now abandoned, the substance of which is fully incorporated herein by reference.

The substrate may be permanently deformable. In such cases, the substrate may have a yield point and thickness such that the substrate substantially conforms to the shape of a tooth and its adjoining soft tissue via permanent deformation under a pressure less than about 250,000 Pascals. The permanently deformable substrate that is preferred has visco-elastic properties that enable it to creep as well as bend in order to conform across several teeth and around the arch of the wearer's mouth. It is important that the necessary permanent deformation occur under minimum normal force being applied by the wearer.

In some embodiments, the substrate 22 may have a relatively low flexural stiffness so as to enable it to drape over the contoured surfaces of the teeth with very little force being exerted; that is, conformity to the curvature of the wearer's mouth, teeth, and gaps between teeth is maintained because there is little residual force within the substrate to cause it to return to its substantially flat shape. The flexibility of the substrate enables it to contact adjoining soft tissue over an extended period of time without physical irritation. The substrate may not require pressure to form it against the teeth and can be readily conformable to the tooth surfaces and the interstitial tooth spaces without permanent deformation when it is applied. When the substrate 22 is provided in the form of a thin, flexible strip, the substrate may have a thickness of at least about 0.001 mm or at least about 0.005 mm and/or less than about 1 mm, or less than about 0.1 mm, or less than about 0.05 mm, or less than about 0.03 mm, or less than about 0.02 mm.

Flexural stiffness is a material property that is a function of a combination of substrate thickness, width, and material modulus of elasticity. In a preferred embodiment but not required for the present invention, the flexible substrate has a flexural stiffness of less than about 5 grams/cm as measured on a Handle-O-Meter, model #211-300, available from Thwing-Albert Instrument Co. of Philadelphia, Pa., as per test method ASTM D2923-95. This test is a method for measuring the rigidity of polyolefin film and sheeting. It determines the resistance to flexure of a sample by using a strain gauge affixed to the end of a horizontal beam. The opposite end of the beam presses across a strip of the sample to force a portion of the strip into a vertical groove in a horizontal platform upon which the sample rests. A microammeter, wired to the strain gauge is calibrated in grams of deflection force. The rigidity of the sample is read directly from the microammeter and expressed as grams per centimeter of sample strip width. Preferably, the substrate 22 has a flexural stiffness less than about 4 grams/cm, or less than about 3 grams/cm, or between about 0.1 grams/cm and about 1 grams/cm.

The substrate may be held in place on the teeth by adhesive attachment provided by the oral care composition. Any adhesiveness is not supplied by wax. The viscosity and general tackiness of the oral care composition can cause the substrate to be adhesively attached to the oral surface without substantial slippage from the frictional forces created by the lips, teeth, tongue and other oral surfaces rubbing against the substrate while talking, drinking, etc. However, this adhesion to the oral surface is low enough to allow the substrate to be easily removed by the wearer by simply peeling off the substrate using one's finger, fingernail or rubbing with a soft implement such as a cotton balls and swabs or gauze pads. The delivery system is easily removable from the oral surfaces without the use of an instrument, a chemical solvent or agent, or excessive friction. The chemical solvents include organic solvent known for use in the oral cavity such as alcohols, and other safe solvents such as water, that can be used to dilute the gelling agent.

A peel force that may be used to remove the substrate from the oral surface is from about 1 gram to about 50 grams for a 1.5 cm substrate width (approximately 17 grams/cm) is all that is required. Preferably, the peel force is from about 5 grams to about 40 grams and more preferably from about 20 grams to about 30 grams. The low peel force is desired for consumer handling purposes. The low peel force is possible because of the non-aggressive nature of the oral care composition necessary to adhere the substrate having lower flexural stiffness. That is, a substrate having high flexural stiffness would require an aggressive adhesive to stop the substrate from pulling it away from the contours of the oral surface it is attached to.

In some embodiments, the substrate may be nonbioerodible, while in others, it may be bioerodible.

### The Release Liner

As discussed above, a removable release liner 32 can be optionally provided adjacent the oral care composition layer 28. The release liner 32 can be formed from any material which exhibits less affinity for the oral care composition layer 28 than the oral care composition layer 28 exhibits for itself and for the substrate 22. For example, the release liner 32 can be formed from paper or a polyester, such as SCOTCHPAK® which is manufactured by the 3M Corp. of Minneapolis, Minn., which is coated with a non-stick material in order to aid release of the tooth whitening composition from the release liner 32 when the substrate 22 is pulled away from the release liner 32. Exemplary coatings can include wax, silicone, fluoropolymers such as Teflon®, fluorosilicones, or other non-stick type materials. Also, suitable coatings might include one of the coatings described in U.S. Pat. Nos. 3,810,874; 4,472,480; 4,567,073; 4,614,667; 4,830,910; and 5,306,758, the substances of which are incorporated herein by reference. A further description of materials suitable which might be suitable as release agents is found in Kirk-Othmer Encyclopedia of Chemical Technology, Fourth Edition, Volume 21, pp. 207-218, incorporated herein by reference. While the release liner 32 should be at least the same size and shape as the substrate 22 as shown in FIG. 1, the release liner 32 can extend beyond the substrate so that it is easier to remove the substrate 22 (and the attendant first and second layers) from the release liner 32.

### Delivery Layer

In accordance with one aspect of the present invention, the delivery layer 30 comprises a microcrystalline wax.

A “wax” has traditionally referred to a substance that is secreted by bees (beeswax) and used by them in constructing their honeycombs. As a general term, a wax is understood to be a substance with properties similar to beeswax, namely plastic (malleable) at normal ambient temperatures, a melting point above approximately 45 degrees Celsius (113 degree Fahrenheit), a relatively low viscosity when melted (unlike many plastics), insoluble in water, and hydrophobic. The various materials named waxes do not form a chemically homogeneous group. They are made up of various substances including hydrocarbons (normal or branched alkanes and alkenes), ketones, diketones, primary and secondary alcohols, aldehydes, sterol esters, alkanoic acids, terpenes (squalene) and monoesters (wax esters), all with long or very long carbon chains (from 12 up to about 38 carbon atoms) and solid in a large range of temperature.

Different types of waxes include animal and insect waxes (beeswax, Chinese wax, shellac wax, spermaceti, lanolin), vegetable waxes (bayberry wax, candelilla wax, carnauba wax, castor wax, esparto wax, Japan wax, jojoba oil, ouricury wax, rice bran wax), mineral waxes (cresin waxes, montan wax, ozocerite, peat waxes), petroleum waxes (paraffin wax, microcrystalline wax), and synthetic waxes (polyethylene waxes, Fischer-Tropsch waxes, chemically modified waxes, substituted amide waxes, polymerized α-olefins).

In some embodiments of the present invention, the delivery layer comprises both microcrystalline wax and paraffin wax. While microcrystalline wax and paraffin wax are both petroleum waxes, there are specific differences between them. Microcrystalline wax is a refined mixture of solid, saturated aliphatic hydrocarbons produced by de-oiling certain fractions from the petroleum refining process. In contrast to the more familiar paraffin wax which contains mostly unbranched alkanes, microcrystalline wax contains a higher percentage of isoparaffinic (branched) hydrocarbons and naphthenic hydrocarbons. It is characterized by the fineness of its crystals in contrast to the larger crystal of paraffin wax. It consists of high molecular weight saturated aliphatic hydrocarbons. It is generally darker, more viscous, denser, tackier and more elastic than paraffin waxes, and has a higher molecular weight and melting point. The elastic and adhesive characteristics of microcrystalline waxes are related to the non-straight chain components which they contain. Typical microcrystalline wax crystal structure is small and thin, making them more flexible than paraffin wax.

As a general statement not meant to be limiting, microcrystalline waxes may be broadly divided into three categories. Type 1 is a laminating grade, with a melting point from 70 to 76 degrees Celsius and a hardness from 20 to 35 dmm. Type 2 is a coating grade, with a melting point from 76 to 85 degrees Celsius and a hardness from 14 to 25 dmm. Type 3 is a hardening grade, with a melting point from 85 to 95 degrees Celsius and a hardness from 6 to 14 dmm.

In some embodiments of the present invention, the ratio, by weight, of microcrystalline wax to paraffin wax ranges from about 49:1 to about 1:1. In other embodiments, the ratio, by weight, of microcrystalline wax to paraffin wax ranges from about 9:1 to about 3:2. In still other embodiments, the ratio, by weight, of microcrystalline wax to paraffin wax ranges from about 4:1 to about 7:3.

Typically, the paraffin wax of the present invention has a melting point from about 45 to about 69 degrees Celsius, or from about 55 to about 69 degrees Celsius, or from about 60 to about 69 degrees Celsius, as determined under ASTM test method D 87-07. The microcrystalline wax of the present invention has a melting point from about 70 to about 95 degrees Celsius, or from about 72 to about 90 degrees Celsius, or from about 75 to about 88 degrees Celsius, as determined under ASTM test method D 127-05. Paraffin wax and microcrystalline wax are described in more detail in Hawley's Condensed Chemical Dictionary, Thirteenth Edition, Revised by Richard J. Lewis, Sr., published by John Wiley & Sons, Inc. (1997), incorporated herein by reference in its entirety.

The needle penetration (a measure of hardness as determined under ASTM test method D 1321-04) of the paraffin wax can be from about 5 to about 40 dmm (decimillimeter), or from about 9 to about 20 dmm, or from about 10 to about 16 dmm. The needle penetration of the microcrystalline wax can be from about 5 to about 50 dmm, or from about 10 to about 25 dmm, or from about 14 to about 20 dmm. The molecular weight of the paraffin wax can be from about 114 g/mol to about 844 g/mol with from about 8 to about 60 carbons in the backbone, or from about 212 g/mol to about 634 g/mol with about 15 to about 45 carbons in the backbone, or from about 282 g/mol to about 564 g/mol with about 20 to about 40 carbons in the backbone.

Other waxy substances, generally categorized as lipids, may be incorporated into the delivery layer. Some examples include those classes of ingredients described as fatty alcohols, fatty aldehydes, fatty acids, derivatives of fatty acids including tri-, di-, and monoglycerides, phospholipids, and cholesterols. Fatty alcohols are aliphatic alcohols, saturated or unsaturated, derived from natural fats and oils; examples include but are not limited to: cetyl alcohol, palmitoleyl alcohol, stearyl alcohol, isostearyl alcohol, or behenyl alcohol. Fatty acids are aliphatic carboxylic acids and can be either saturated or unsaturated; examples include but are not limited to: stearic acid, lauric acid, palmitic acid, oleic acid, or linoleic acid. Derivatives of fatty acids are based upon esterfication of glycerol with one, two, or three fatty acids. These fatty acids may or may not be the same as each other and are similar to those described above.

In some embodiments, in addition to either microcrystalline wax alone or a mixture of microcrystalline wax and paraffin wax, any delivery layer may also comprise one or more aesthetic agents or other oral care agents. The one or more aesthetic agents can be provided in the form of a viscous liquid, a paste, a gel, a solution, a solid, a powder, or any other state or phase that can form a layer. As used herein, the phrase “aesthetic agent” refers to any agent that affects the gustatory, olfactory, or somatosensory sensations. Examples of aesthetic agents include flavoring agents (e.g., sweetening agents, bitter agents, sour agents, etc.), aromatic agents (e.g., volatile oils and essences), and sensate agents (e.g., cooling agents, warming agents, etc.). These agents may be encapsulated, as discussed more fully hereafter, to target delivery of the agents or to protect the agents from reactive substances such as water, peroxide, and other constituents of the tooth whitening product. Some suitable agents are described hereafter. While these agents have been described herein as flavoring agents, aromatic agents, and sensate agents for convenience, it will be appreciated that some agents may be classified in more than one category. For example, peppermint oil may be considered both an aromatic agent and a sensate agent (i.e., a cooling agent).

Also, a plurality of aesthetic agents can be incorporated into the delivery layer 30. For example, it may be desirable to include a sweetener, such as sucralose, in combination with other aesthetic agents, such as menthol monophosphate (MMP) and N-ethyl-p-menthan-3-carboxamide (WS-3). In some embodiments, such as in FIG. 10, there may be more than one delivery layer, wherein the first delivery layer 30 comprises a microcrystalline wax and an aesthetic agent and the second delivery layer 44 comprises a sweetening agent, such as sucralose, for example. The first delivery layer 30 may be disposed adjacent the second side of the substrate 26, while the second delivery layer 44 may be disposed adjacent the first delivery layer 30.

Suitable sweeteners include those well known in the art, including both natural and artificial sweeteners. Some suitable sweeteners include monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin. Suitable artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2, 2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include Dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like. Sweeteners derived from naturally occurring sweeteners, such as a chlorinated derivative of ordinary sugar (sucrose), known, for example, under the product description of sucralose as well as protein based sweeteners such as thaumatoccous danielli (Thaumatin I and II) can be used. In general, the composition forming the second layer 30 can contain greater than about 0%, or greater than about 0.001%, or greater than about 0.01%, or greater than about 0.5%, or greater than about 1%, or greater than about 10% or greater than about 25% or greater than about 50% and/or less than about 99%, or less than about 90%, or less than about 40%, or less than about 20%, or less than about 5%, or less than about 3% by weight of a sweetening agent. The upper sweetener range is generally applicable when the second layer 30 substantially comprises a sweetening agent in the form of a powder, as discussed more fully hereafter.

Cooling agents can be selected from any of a wide variety of materials. Included among such materials are carboxamides, menthol, ketals, diols, and mixtures thereof. Preferred cooling agents in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred cooling agents are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosures of which are incorporated herein by reference. In general, the composition forming the second layer 30 can contain greater than about 0%, or greater than about 0.001%, or greater than about 0.01%, or greater than about 0.1%, or greater than about 1%, or greater than about 10%, or greater than about 25%, or greater than about 50% and/or less than about 99%, or less than about 90%, or less than about 75%, or less than about 40%, or less than about 10%, or less than about 5%, or less than about 2% by weight of a cooling agent. The upper sweetener range is generally applicable when the second layer 30 substantially comprises a cooling agent in the form of a powder, as discussed more fully hereafter.

Natural and artificial aromatic agents can be used. Some suitable aromatic agents include synthetic oils, essential oils, oleo resins, essences, and extracts derived from plants, leaves, flowers, fruits and so forth, and combinations thereof. Representative oils include spearmint oil, cinnamon oil, oil of wintergreen, peppermint oil, clove oil, bay oil, eucalyptus oil, thyme oil, cedar leaf oil, oil of nutmeg, oil of sage, oil of bitter almonds. Also useful are artificial and natural fruit oils and essences, including vanilla, citrus, lemon, orange, grape, lime and grapefruit oils and fruit essences including apple, pear, peach, strawberry, raspberry, cherry, plum, pineapple, apricot, and so forth. These aromatic agents may be used individually or in admixture. Commonly used aromatic agents include mints (e.g., peppermint, menthol, spearmint, and wintergreen), cinnamon derivatives and various fruit oils or essences whether employed individually or in admixture. In general, the composition forming the second layer 30 can contain greater than about 0%, or greater than about 0.001%, or greater than about 0.01%, or greater than about 0.1%, or greater than about 1% and/or less than about 60%, or less than about 30%, or less than about 15%, or less than about 5% by weight of an aromatic agent.

The aesthetic agents can also be combined, bound, or complexed with other elements and/or encapsulated. For example, a composition may be formulated by phosphorylating at least one aesthetic agent, such as an aromatic agent. These compositions are referred to herein as phosphate derivatives and are described more fully in WO 95/07683, the substance of which is incorporated herein by reference. Phosphate derivatives also include linking at least one aesthetic agent to an adherent component via a phosphate bridge. Pyrophosphate and triphosphate groupings may be substituted for the phosphate group. As used herein, the term “adherent component” is intended to refer to either monomers, oligomers, or polymers having hydroxy, amino, or thiol functionalities which are capable of forming either ester amido, or thioester linkages with phosphorus (V) atoms. The monomers, oligomers, or polymers may also posses additional hydroxy, amino, or thiol groups which may either remain unsubstituted or be linked via ester amido, or thioester linkages to a phosphorus (V) atom which is also attached to the aesthetic agent. Preferred compounds are selected from the group consisting of C12-C18 diacyl glycerol, partially hydrolized vinyl acetate/ethylene copolymer, cellulose, chitin, glucose, glucosamine, silica gel, gycerol, and lower alkyl vinyl ether-maleic acids. The aesthetic agent may also be linked to phosphorous via two functional groups or attachment sites or bound via Coulombic interaction with charged compounds or materials, including polymers.

The aesthetic agent of the phosphate derivative can be released after cleavage of the phosphate from the aesthetic agent by phosphatase enzymes, such as those commonly found in the oral cavity. The phosphatase enzymes include but are not limited to acid, basic, and pyrophosphatases. Preferred aesthetic agents including cooling agents selected from the group consisting of menthol, 3-1-menthoxypropane-1, 2-diol (“TK-10”), menthone glycercol acteal (“MGA”), and menthyl lactate. The terms “menthol” and “menthyl” as used herein include dextro- and levororatory isomers of these compounds and reacemic mixtures thereof. Preferred phosphate derivatives include menthyl monophosphate, menthol monophosphate, eugenyl monophosphate, thymyl monophosphate, 1-menthyl diphosphate, bis 1-menthyl pyrophosphate, and 1-mehtyl triphosphate. The phosphate derivatives can be represented by the following formula:

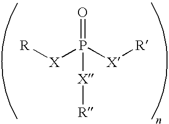

In the above formula,

R is selected is preferably selected from the group consisting of a cooling agent, a sweetening agent, and a flavoring agent;

R′ and R″ are independently selected from the group consisting of R, an adherent component, M+, M++, C+, and hydrogen;

M+and M++ are metal cations that are significant to the organic or bodily processes of a human. Preferred M+ cations are sodium and potassium. Preferred M++ cations are zinc, magnesium, manganese, copper, and stannous.

C+ is an organic cation. An organic cation contains positively charged nitrogen, phosphorous, oxygen, or sulfur atoms. Such cations may contain more than one positively-charged site and in the case of oligomers or polymers containing nitorgen, phosphorous, oxygen, or sulfur atoms, many positively-charged centers may exist. Preferred organic cations include, ammonium, protonated amines such as protonated glucosamine, and partially or fully protonated amine-containing polymers such as protonate chitosan.

X, X′, and X″ are independently selected from the group consisting of oxygen, nitrogen, and sulfur; and

n is an integer from 1 to 3.

In addition, R′ may equal R″, preferably wherein R′ and R″ are selected from the group consisting of calcium, zinc, and magnesium, manganese, copper, and stannous. Because the phosphate derivatives are highly stable and release the aesthetic agent only after cleavage of the phosphate from the aesthetic agent, phosphate derivatives are particularly preferred in the present invention.

The aesthetic agents may also be encapsulated in order to increase the stability of the aesthetic agent. Suitable encapsulation agents include any of the known cyclodextrins, such as unsubstituted cyclodextrins containing from six to twelve glucose units, especially, alpha-, beta-, gamma-cyclodextrins, and mixtures thereof, and/or their derivatives, and/or mixtures thereof, that are capable of forming inclusion complexes with the above-described sensating agents. Alpha-, beta-, and gamma-cyclodextrins can be obtained from, among others, American Maize-Products Company (Amaizo), Hammond, Ind.; Roquette Corporation, Gurnee, Ill.; and Chinoin Pharmaceutical and Chemical Works, Ltd., Budapest, Hungary. There are many derivatives of cyclodextrins that are known. Representative derivatives include those disclosed in U.S. Pat. No. 3,426,011, Parmerter et al., issued Feb. 4, 1969; U.S. Pat. Nos. 3,453,257, 3,453,258, 3,453,259, and 3,453,260, all in the names of Parmerter et al., and all issued Jul. 1, 1969; U.S. Pat. No. 3,459,731, Gramera et al., issued Aug. 5, 1969; U.S. Pat. No. 3,553,191, Parmerter et al., issued Jan. 5, 1971; U.S. Pat. No. 3,565,887, Parmerter et al., issued Feb. 23, 1971; U.S. Pat. No. 4,535,152, Szejtli et al., issued Aug. 13, 1985; U.S. Pat. No. 4,616,008, Hirai et al., issued Oct. 7, 1986; U.S. Pat. No. 4,638,058, Brandt et al., issued Jan. 20, 1987; U.S. Pat. No. 4,746,734, Tsuchiyama et al., issued May 24, 1988; and U.S. Pat. No. 4,678,598, Ogino et al., issued Jul. 7, 1987, all of said patents being incorporated herein by reference. Examples of cyclodextrin derivatives suitable for use herein include methyl-β-CD, hydroxyethyl-β-CD, and hydroxypropyl-β-CD of different degrees of substitution (DS), available from, among others, Aldrich Chemical Company, Milwaukee, Wis.; Wacker Chemicals (USA), New Canaan, Conn.; and Chinoin Pharmaceutical Works, Budapest, Hungary. Water-soluble derivatives are also highly desirable.

The individual cyclodextrins can also be linked together, e.g., using multifunctional agents to form oligomers, polymers, etc. Examples of such materials are available commercially from Amaizo and from Aldrich Chemical Company (β-CD/epichlorohydrin copolymers). It may also be desirable to use mixtures of cyclodextrins to provide a mixture of complexes. Mixtures of cyclodextrins can conveniently be obtained by using intermediate products from known processes for the preparation of cyclodextrins including those processes described in U.S. Pat. No. 3,425,910, Armbruster et al., issued Feb. 4, 1969; U.S. Pat. No. 3,812,001, Okada et al., issued May 21, 1974; U.S. Pat. No. 4,317,881, Yagi et al., issued Mar. 2, 1982; U.S. Pat. No. 4,418,144, Okada et al., issued Nov. 29, 1983; and U.S. Pat. No. 4,738,923, Ammeraal, issued Apr. 19, 1988, all of said patents being incorporated herein by reference. Some cyclodextrin mixtures are commercially available from, e.g., Ensuiko Sugar Refining Company, Yokohama, Japan. The cyclodextrin complexes can be formed in any of the ways know in the art. Examples of such processes are described in U.S. Pat. Nos. 5,571,782; 3,812,011; 4,317,881; 4,418,144; and 4,378,923, the substances of which are incorporated herein by reference.

Other encapsulation technologies may also be used, such as microcapsules that comprise a core formed from an aesthetic agent and a coating layer over the core to control the release of the aesthetic agent. Coating materials that are water resistant and that release the aesthetic agent during use are preferred, including coating materials that fracture under physical forces such as chewing or that disperse or emulsify when contacted by saliva. Suitable coatings include those formed from gelatin, carboxymethyl cellulose, gum arabic, casein, alginate, waxes, lipids, and mixtures thereof. The coating layer can be prepared by coacervation which is a process for the aggregation of colloidal spheres held together by electrostatic forces and can be carried out by diluting an emulsion of the flavor oil in the presence of such colloidal materials with water, adjusting the pH of the emulsion or the temperature, or by any combination such techniques. U.S. Pat. Nos. 5,759,599; 5,266,335; 5,498,439; and 4,983,404; the substances of which are incorporated herein by reference, describe some encapsulation coatings and processes for forming the same that can be used with the present invention. Other processes known in the art can also be used (e.g., spray coating). Multiple encapsulations can be used with the present invention. For example, different encapsulation techniques could be used with different aesthetic agents that are both incorporated into the delivery layer 30 to provide differing release characteristics. Alternatively, several encapsulation techniques could be used to provide multiple layers of encapsulation about a single aesthetic agent.

Any delivery layer may further, or alternatively, comprise at least one oral care active. For example, phosphates, fluoride ion sources, anti-microbial agents, anti-inflammatory agents, nutrients, enzymes, anti-oxidants, H-2 antagonists, and so forth may be used as an oral care active. These and other oral care actives that are suitable for use with the present invention are described in U.S. Pat. No. 6,136,297, the substance of which is incorporated herein by reference.

### Method of Manufacture

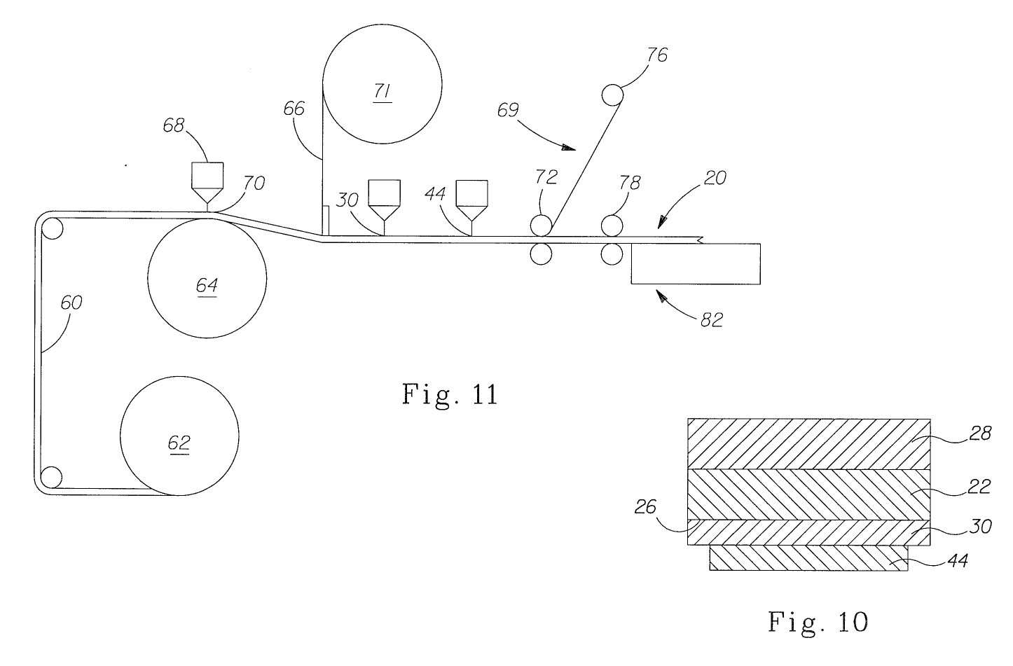

Referring to FIG. 11, one method for forming the oral care delivery system 20 will now be described. A sheet 60 of the release liner is unrolled from the roller 62 and is fed over drum 64. The sheet 60 of the release liner (as well as sheet 66 of the substrate 22) may be formed by several of the film making processes known in the art. The sheets 60 and 66 can be made by a blown process or a cast process. Processes, such as extrusion and other processes that do not affect the flexural rigidity of the substrate may also be used. A nozzle 68 applies (by slot, spray, roll transfer, or any other suitable technique) the oral care composition layer 70 onto the sheet 60 of the release liner. The sheet 66 of the substrate 22 is unrolled from the roller 71 and lightly pressed onto the oral care composition layer 70, thereby forming a three layer laminate. The laminate is fed to the rollers 72 which cut the outer edge of the substrate 22. After the cutting operation at rollers 72, the excess sheet 69 of the substrate 22 is taken up by the roller 76, thereby leaving the substrate 22 and the oral care composition layer on the sheet 60 of the release liner. The rollers 78 cut the release liner to form individual tooth whitening products 20. The tooth whitening products 20 are collected by the conveyor 82, after which the tooth whitening products 20 can be inserted into a package to form a packaged tooth whitening product.

The first delivery layer 30 can be formed before or after the formation of the oral care composition layer on the sheet 60. The first delivery layer can be formed by admixing the aesthetic agent, the microcrystalline wax, paraffin wax, and any other desired constituents (e.g., a humectant such as glycerol or propylene glycol). The mixture can be sprayed, via a nozzle, or otherwise coated onto the sheet 66 that is used to form the substrate. A second delivery layer 44 can be applied adjacent to the first delivery layer by first admixing the desired constituents, sweetener and water. The solution or mixture can be sprayed or otherwise applied to the first delivery layer.

### Method of Use

To practice the invention, the substrate is applied by the consumer to a plurality of adjacent teeth. The side of the substrate facing the teeth is coated with a tooth whitening composition that is preferably in a viscous state to provide not only the tooth whitening agent but also tackiness between the tooth surfaces and the substrate to hold the substrate in place for an extended period of time. The substrate may optionally be applied to the soft tissue adjacent the teeth and may also optionally be folded over the incisal edges of the plurality of teeth and onto their lingual sides. The substrate may readily conform to the teeth by lightly pressing it against the teeth and/or by the consumer gently sucking through the gaps between the teeth. The substrate may be easily removed by the wearer by peeling it off. Preferably, each successive treatment will use a fresh tooth whitening product. Because the delivery layer containing the aesthetic agent is directly exposed to the oral cavity (i.e., it is exposed to the lips and/or tongue during use), saliva can readily liberate the aesthetic agent thereby providing a pleasurable oral experience. The liberation of the aesthetic agent can be facilitated by movement of the tip of the tongue over the exposed surface of the delivery layer on one or both of the labial and lingual sides of the substrate when it is folded over the incisal edges of the teeth.

The tooth surface is not required to be prepared before the tooth whitening product is applied. For example, the wearer may or may not choose to brush his teeth or rinse his mouth before applying the delivery system. The surfaces of the teeth are not required to be dried or to be excessively wet with saliva or water before the substrate is applied. Preferably, the substrate and compositions are substantially transparent so as to be almost unnoticeable when wom. Thinness of the tooth whitening product enables the higher temperature inside of the wearer's mouth to conduct heat through the substrate to the normally cooler teeth in order to accelerate the rate of diffusion of the tooth whitening agent into the surfaces of the teeth. Preferably, the wearer applies the oral care delivery system to the teeth continuously for about 5 minutes to about 120 minutes a day, preferably from about 30 minutes to about 60 minutes. Generally, this is done once a day for about 7 to 28 consecutive days. The amount of time and the number of days are dependent upon several factors, including the amount of bleaching desired, the wearer's teeth, and if initial or maintenance bleaching is desired.

### EXAMPLES

The following examples of the oral care delivery system, oral care composition layer, first delivery layer, and second delivery layer further describe and demonstrate embodiments within the scope of the present invention. These examples are given solely for the purpose of illustration and are not to be construed as limitations of the present invention as many variations thereof are possible without departing from the spirit and scope of the present invention. Percentages herein are by weight unless otherwise stated.

GEL EXAMPLES - ORAL CARE DELIVERY SYSTEM

| | 1 | 2 | 3 | 4 | 5 | 6 |
|---|---|---|---|---|---|---|
| Oral Care Composition | 60.776% | 72.157% | 74.857% | 63.379% | 66.246% | 80.000% |
| Layer | | | | | | |
| First Delivery Layer | 30.966% | 18.039% | 14.972% | 32.292% | 33.754% | 20.000% |
| Second Delivery Layer | 8.258% | 9.804% | 10.171% | 4.329% | — | — |

GEL EXAMPLES - ORAL CARE COMPOSITION LAYER

| | 7 | 8 | 9 | 10 | 11 | 12 |
|---|---|---|---|---|---|---|
| Glycerin | 10.000% | 10.000% | 20.000% | 10.000% | — | — |
| Water | 67.776% | 64.348% | 54.348% | 64.248% | 74.148% | 67.776% |
| Hydrogen Peroxide | 15.143% | 18.571% | 18.571% | 8.571% | 18.571% | 15.143% |
| (35% solution) | | | | | | |
| Carboxypolymethylene | 4.500% | 4.500% | 4.500% | 4.500% | 4.500% | 4.500% |
| Sodium Hydroxide | 2.000% | 2.000% | 2.000% | 2.000% | 2.000% | 2.000% |
| (50% solution) | | | | | | |
| Sodium Saccharin | — | — | — | 0.100% | 0.200% | — |
| Sodium Stannate | 0.200% | 0.200% | 0.200% | 0.200% | 0.200% | 0.200% |
| Sodium Pyrophosphate | 0.381% | 0.381% | 0.381% | 0.381% | 0.381% | 0.381% |
| Propylene Glycol | — | — | — | — | — | 10.000% |
| Pluronic 407 | — | — | — | — | — | — |
| | 13 | 14 | 15 | 16 | 17 | 18 |
| Glycerin | 10.000% | — | 3.000% | 15.000% | 10.000% | 10.000% |
| Water | 68.157% | 57.276% | 72.576% | 63.076% | 72.919% | 66.954% |
| Hydrogen Peroxide | 15.143% | 15.143% | 17.143% | 15.143% | — | 17.143% |
| (35% solution) | | | | | | |
| Carboxypolymethylene | 4.500% | — | 4.500% | 4.500% | 4.500% | 4.500% |
| Sodium Hydroxide | 2.000% | 2.000% | 2.200% | 1.700% | 2.000% | — |
| (50% solution) | | | | | | |
| Sodium Saccharin | — | — | — | — | — | — |
| Sodium Stannate | 0.200% | 0.200% | 0.200% | 0.200% | 0.200% | — |
| Sodium Pyrophosphate | — | 0.381% | 0.381% | 0.381% | 0.381% | — |
| Propylene Glycol | — | — | — | — | — | — |
| Pluronic 407 | — | 25.000% | — | — | — | — |
| Potassium Hydroxide | — | — | — | — | — | 1.403% |
| Carbamide Peroxide | — | — | — | — | 10.000% | — |

WAX EXAMPLES - FIRST DELIVERY LAYER

| | 19 | 20 | 21 | 22 | 23 | 24 |
|---|---|---|---|---|---|---|
| Microcrystalline wax | 48.750% | 40.000% | 40.000% | 48.750% | 50.000% | 52.500% |
| (mp 79.4-86.7° C.) | | | | | | |
| Microcrystalline wax | — | — | — | — | — | — |
| (mp 76.7-82.2° C.) | | | | | | |
| Paraffin wax | 16.250% | 20.000% | — | — | — | — |
| (mp 61.1-64.4° C.) | | | | | | |
| Paraffin wax | — | — | 20.000% | 16.250% | 15.000% | 17.500% |
| (mp 66.7-69.4° C.) | | | | | | |
| Hydrogenated castor oil | — | — | — | — | — | — |
| Hydrogenated vegetable | — | — | — | — | — | — |
| oil | | | | | | |
| Cetyl alcohol | — | — | — | — | — | — |
| Flavor | 35.000% | 40.000% | 40.000% | 35.000% | 35.000% | 30.000% |
| | 25 | 26 | 27 | 28 | 29 | 30 |
| Microcrystalline wax | 60.000% | 40.000% | 85.000% | 60.000% | 65.000% | 90.000% |
| (mp 79.4-86.7° C.) | | | | | | |
| Microcrystalline wax | — | — | — | 10.000% | 10.000% | — |
| (mp 76.7-82.2° C.) | | | | | | |
| Paraffin wax | — | — | — | — | — | — |
| (mp 61.1-64.4° C.) | | | | | | |
| Paraffin wax | — | — | — | — | — | — |
| (mp 66.7-69.4° C.) | | | | | | |
| Hydrogenated castor oil | — | — | — | 5.000% | — | — |
| Hydrogenated vegetable | — | — | — | 5.000% | 5.000% | — |
| oil | | | | | | |
| Cetyl alcohol | — | 20.000% | — | 5.000% | 5.000% | — |
| Flavor | 40.000% | 40.000% | 15.000% | 15.000% | 15.000% | 10.000% |

GEL EXAMPLES - SECOND DELIVERY LAYER

| | 31 | 32 | 33 | 34 | 35 | 36 |
|---|---|---|---|---|---|---|
| Water | 89.500% | 89.500% | 89.500% | 89.500% | — | 92.500% |
| Hydrogen Peroxide | 3.000% | 3.000% | 3.000% | 3.000% | — | — |
| (35% solution) | | | | | | |
| Sucralose | 7.500% | — | — | 2.500% | 100.000% | 7.500% |
| Saccharin | — | 7.500% | — | 2.500% | — | — |
| Acesulfame K | — | — | 7.500% | 2.500% | — | — |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govem.

While particular embodiments of the present invention have been illustrated and described, it will be obvious to those skilled in the art that various changes and modifications may be made without departing from the spirit and scope of the invention. It is therefore intended to cover in the appended claims all such changes and modifications that are within the scope of the invention.

What is claimed is

1. An oral care delivery system comprising:
a. a flexible substrate, said substrate having a first side and a second side;
b. at least one oral care composition, wherein the at least one oral care composition is disposed adjacent said first side of said substrate; and
c. at least one delivery layer, wherein a first delivery layer is disposed adjacent said second side of said substrate and wherein said first delivery layer comprises a microcrystalline wax having a melting point from about 70 to about 95 degrees Celsius;
wherein the oral care delivery system is applied to the surfaces of a plurality of teeth.

2. The oral care delivery system of claim 1, wherein said first delivery layer further comprises paraffin wax.

3. The oral care delivery system of claim 1, wherein said oral care composition is a tooth whitening agent.

4. The oral care delivery system of claim 1, wherein the first delivery layer further comprises an aesthetic agent.

5. The oral care delivery system of claim 1, wherein the first delivery layer is nonaqueous.

6. The oral care delivery system of claim 1, wherein the first delivery layer further comprises at least one oral care active.

7. The oral care delivery system of claim 3, wherein the microcrystalline wax is compatible with the tooth whitening agent.

8. The oral care delivery system of claim 1, wherein the microcrystalline wax has a melting point from about 75 to about 88 degrees Celsius.

9. The oral care delivery system of claim 2, wherein the ratio, by weight, of microcrystalline wax to paraffin wax is from about 49:1 to about 1:1.

10. The oral care delivery system of claim 2, wherein the ratio, by weight, of microcrystalline wax to paraffin wax is from about 9:1 to about 3:2.

11. The oral care delivery system of claim 2, wherein the ratio, by weight, of microcrystalline wax to paraffin wax is from about 4:1 to about 7:3.

12. The oral care delivery system of claim 3, wherein said tooth whitening agent is a peroxide agent.

13. The oral care delivery system of claim 4, wherein said aesthetic agent is sucralose.

14. The oral care delivery system of claim 4, wherein the first delivery layer further comprises a flavoring component, and wherein the first delivery layer consists of about 60% wax and about 40% flavoring component.

15. The oral care delivery system of claim 4, wherein the first delivery layer further comprises a flavoring component, and wherein the first delivery layer consists of about 65% wax and about 35% flavoring component.

16. The oral care delivery system of claim 4, wherein the first delivery layer further comprises a flavoring component, and wherein the first delivery layer consists of about 70% wax and about 30% flavoring component.

17. The oral care delivery system of claim 4, further comprising a second delivery layer disposed adjacent the first delivery layer, wherein said second delivery layer comprises a sweetening agent.

18. The oral care delivery system of claim 17, wherein said sweetening agent is sucralose.

19. A method of delivering at least one oral care composition to a surface of a tooth and its adjoining soft tissue, said method comprising the steps of:
a. applying the at least one oral care composition onto a first side of a flexible substrate;
b. applying at least one delivery layer onto a second side of the flexible substrate, wherein a first delivery layer comprises a microcrystalline wax having a melting point between 70 and 95 degrees Celsius; and
c. applying said flexible substrate onto said surface of a tooth and its adjoining soft tissue.

20. A method of bleaching a plurality of adjacent teeth and delivering an aesthetic agent, comprising applying an oral care delivery system to a plurality of adjacent teeth, wherein the oral care delivery system comprises a flexible substrate, at least one oral care composition, and at least one delivery layer, said method comprising the steps of:
a. applying the at least one oral care composition onto a first side of the flexible substrate;
b. applying the at least one delivery layer onto a second side of the flexible substrate, wherein a first delivery layer comprises a microcrystalline wax having a melting point between 70 and 95 degrees Celsius and an aesthetic agent; and
c. applying said flexible substrate onto a plurality of adjacent teeth.
